(19) United States
(12) Patent Application Publication
(10) Pub. No.: US 2011/0033548 A1
(43) Pub. Date: Feb. 10, 2011

(54) DEGRADABLE CROSSLINKED AMINATED DEXTRAN MICROSPHERES AND METHODS OF USE

(75) Inventors: Cheng-Yu LAI, West Grove, PA (US); Daniela Rodica Radu, West Grove, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/849,035

(22) Filed: Aug. 3, 2010

Related U.S. Application Data
(60) Provisional application No. 61/231,355, filed on Aug. 5, 2009.
(60) Provisional application No. 61/231,354, filed on Aug. 5, 2009.

(51) Int. Cl. A61K 9/14; A61K 38/39; A61K 31/721; A61P 9/00

(52) U.S. Cl. 424/499; 514/17.2; 514/59; 428/402

(57) ABSTRACT

Degradable, crosslinked aminated dextran microspheres are described. The microspheres contain aminated dextran crosslinked with a crosslinking agent having two or more functional groups that are capable of reacting with the primary amine groups of the aminated dextran to form covalent bonds. The degradable, crosslinked aminated dextran microspheres may be useful for temporary therapeutic embolization and drug delivery.

### CROSS-REFERENCE TO RELATED APPLICATIONS

This application claims priority under 35 U.S.C. §119 from U.S. Provisional Application Ser. Nos. 61/231,355 and 61/231,354, both filed on Aug. 5, 2009.

### FIELD OF THE INVENTION

The invention relates to the field of therapeutic embolization. Specifically, degradable, crosslinked aminated dextran microspheres are provided which may be useful for temporary therapeutic embolization and drug delivery.

### BACKGROUND OF THE INVENTION

Therapeutic embolization involves the introduction of a material into the vasculature in order to block the blood flow in a particular region. Therapeutic embolization may be used to treat non-cancerous tumors, such as uterine fibroids, and cancerous tumors. Vascular occlusion in the case of tumors may be used to suppress pain, limit blood loss during surgery, or to cause tumor necrosis. In addition, therapeutic embolization may be used to control bleeding caused by conditions such as stomach ulcers, aneurysms, and injury.

Preformed hydrogel microspheres are one type of embolic material; both non-degradable and degradable hydrogel microspheres have been described. Non-degradable hydrogel microspheres have been produced and used in tissue augmentation and embolization. However, a degradable embolic material could enable the administration of a number of different therapies (e.g., drug delivery and surgery) to a site without permanently occluding the site from blood flow. This could lead to more effective therapies and better patient response to treatments.

Various approaches have been used to prepare degradable microspheres for applications such as embolization, drug delivery, and wound dressings. For example, one type of degradable microsphere incorporates degradable crosslinks. As the crosslinks degrade, the microsphere breaks down into soluble polymer chains. In addition, Figuly (copending and commonly owned U.S. Patent Application Publication No. 2009/0041850) describes degradable microspheres that are prepared without the use of a crosslinking agent. Another type of degradable microsphere is prepared from degradable polymers such as poly(lactide-co-glycolide) copolymers, crosslinked proteins, or crosslinked polysaccharides (see for example, Sung et al., U.S. Pat. No. 7,282,220, Kim et al., U.S. Pat. No. 7,309,500, Smith et al., U.S. Pat. No. 5,549,908, and Weissleder et al., U.S. Pat. No. 5,514,379). Many of the polysaccharide-based degradable microspheres are crosslinked with crosslinking agents that are not ideally biocompatible, for example cyanogen bromide or epichlorohydrin.

Therefore, there is a need for microspheres for use in medical therapy that are degradable into small molecules which are readily cleared from the body and are crosslinked with biocompatible crosslinking agents.

### SUMMARY OF THE INVENTION

The present invention provides degradable microspheres formed by crosslinking aminated dextran containing primary amine groups with a biocompatible crosslinking agent.

In one aspect, the present invention is a composition comprising the reaction products obtained by the reaction of:
a) at least one aminated dextran comprising primary amine groups, said dextran having a weight-average molecular weight of about 1,000 to about 1,000,000 Daltons and an amine substitution level of about 1% to about 65%; and
b) at least one crosslinking agent comprising at least two functional groups capable of reacting with the primary amine groups of the aminated dextran;
wherein:
(i) the aminated dextran and said at least one crosslinking agent are crosslinked through covalent bonds formed between the primary amine groups of the aminated dextran and the functional groups of the crosslinking agent; and
(ii) the composition is degradable and in the form of microspheres having a size of about 10 microns to about 750 microns in diameter.

In another aspect, the present invention is a method for embolization in a mammal comprising administering into the vasculature of said mammal a composition comprising:
a) at least one aminated dextran containing primary amine groups, said at least one aminated dextran having a weight-average molecular weight of about 1,000 to about 1,000,000 Daltons and an amine substitution level of about 1% to about 65%; and
b) at least one crosslinking agent containing at least two functional groups capable of reacting with the primary amine groups of the aminated dextran;
wherein:
   (i) said at least one aminated dextran and said at least one crosslinking agent are crosslinked through covalent bonds formed between the primary amine groups of the aminated dextran and the functional groups of the crosslinking agent; and
   (ii) the composition is in the form of degradable, microspheres having a size of about 10 microns to about 750 microns in diameter.

### DETAILED DESCRIPTION

As used above and throughout the description of the invention, the following terms, unless otherwise indicated, shall be defined as follows:

The term “microsphere” refers to a micron size particle which has a high sphericity measurement. The sphericity measurement of a population of microspheres may be in the range of about 80% to about 100%, with 95% being typical. The microspheres are substantially spherical, that is, on average a population of microspheres have a sphericity measurement of greater than about 90%.

The term “degradable” as used herein refers to the property of the crosslinked dextran amine microspheres disclosed herein whereby the microspheres may be biologically broken down, that is degraded, in a living organism to yield low molecular weight degradation products that are soluble in the biologic environment and are small enough to be easily excreted from the body.

The term “aminated dextran containing primary amine groups”, also referred to herein as “dextran amine”, refers to dextran that is derivatized (i.e., chemically modified) to contain primary amine groups.

The term “primary amine group”, as used herein, refers to a neutral amino group having two free hydrogens.

The term “amine substitution level” as used herein, refers to the percent of saccharide rings in dextran that are substituted with a primary amine group.

The term “functional group” as used herein refers to an atom or a group of atoms within a molecule that is capable of reacting with primary amine groups.

The term “covalent bond” as used herein refers to a type of chemical bonding that is characterized by the sharing of pairs of electrons between atoms.

The term “hydrogel” refers to a water-swellable polymeric matrix, consisting of a three-dimensional network of macromolecules held together by covalent crosslinks that can absorb a substantial amount of water to form an elastic gel.

The term “crosslink” refers to a bond or chain of atoms attached between and linking two different polymer chains.

The term “% by weight”, also referred to herein as wt %, refers to the weight percent relative to the total weight of the solution, unless otherwise specified.

The term “embolization suspension” refers to a suspension that contains microspheres and is administered using a catheter and/or needle for embolization treatment.

The meaning of abbreviations used is as follows: “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “mol %” means mole percent, “Da” means Dalton(s), “kDa” means kiloDalton(s), “M” means molarity, “¹H NMR” means proton nuclear magnetic resonance spectroscopy, “ppm” means parts per million, “rpm” means revolutions per minute, “DMSO” means dimethyl sulfoxide.

A reference to “Aldrich” or a reference to “Sigma” means the said chemical or ingredient was obtained from Sigma-Aldrich, St. Louis, Mo.

Disclosed herein are degradable microspheres formed by crosslinking aminated dextran containing primary amine groups with a biocompatible crosslinking agent. The degradable microspheres may be useful as a degradable embolic material and for drug delivery.

### Aminated Dextran

Dextran is a complex, branched polysaccharide that includes many glucose moieties joined together via glycosidic linkages with a variable degree of branching. Dextrans having various average molecular weights are available from commercial sources such as Sigma-Aldrich (Milwaukee, Wis.) and Pharmacosmos A/S (Holbaek, Denmark). Typically, commercial preparations of dextran are a heterogeneous mixture having a distribution of different molecular weights, as well as a variable degree of branching, and are characterized by various molecular weight averages, for example, the weight-average molecular weight (M_{w}), or the number-average molecular weight (Mₙ), as is known in the art. Suitable dextrans for use herein have a weight-average molecular weight of about 1,000 to about 1,000,000 Daltons, more particularly about 3,000 to about 250,000 Daltons, more particularly about 5,000 to about 100,000 Daltons, and more particularly about 10,000 to about 60,000 Daltons.

Aminated dextran containing primary amine groups can be prepared by chemical derivatization of dextran using methods known in the art. For example, a suitable dextran may be reacted with epichlorohydrin in an aqueous solution in the presence of an acid catalyst, such as zinc borofluoride, to form 3-chloro-2-hydroxypropyl dextran, which is subsequently reacted with aqueous ammonia to give the aminated dextran, as described in detail in the Examples herein below. Additionally, aminated dextran can be prepared by oxidizing a suitable dextran using any suitable oxidizing agent, including but not limited to, periodates, hypochlorites, ozone, peroxides, hydroperoxides, persulfates, and percarbonate, to produce an oxidized dextran containing aldehyde groups. Then, the oxidized dextran can be reacted with a diamine, such as hexamethylene diamine, ethylene diamine, propylene diamine, and the like, to form Schiff base linkages. Optionally, the Schiff base linkages may be treated with a reducing agent such as sodium borohydride to form stable carbon-nitrogen bonds. Aminated dextran may also be prepared by reacting dextran with cyanogen bromide, followed by reaction with a diamine. Additionally, aminated dextran can be prepared by the methods described by Kirakossian et al. (U.S. Pat. No. 7,179,660, Example A). Aminated dextrans having different amine substitution levels may be prepared by varying the ratio of the reactants as is known in the art.

The amine substitution level of the aminated dextran may be determined using proton NMR by determining the ratio of the integral of the peaks corresponding to the pendant amine-containing groups to the sum of the integrals of the peaks corresponding to the anomeric protons of the glucose ring and comparing it to the expected ratio for a fully derivatized product.

Suitable aminated dextrans for use herein have a weight-average molecular weight of about 1,000 to about 1,000,000 Daltons, more particularly about 3,000 to about 250,000 Daltons, more particularly about 5,000 to about 100,000 Daltons, and more particularly about 10,000 to about 60,000 Daltons, and an amine substitution level of about 1% to about 65%, more particularly about 1% to about 40%, more particularly about 1% to about 5%, and more particularly about 2% to about 3%.

### Crosslinking Agents

Suitable crosslinking agents for use in the preparation of the degradable microspheres disclosed herein contain at least two functional groups that are capable of reacting with the primary amine groups of the aminated dextran to form a crosslinked network. Functional groups that are capable of reacting with primary amine groups include, but are not limited to, electrophilic groups such as aldehyde, ketone, glyoxal, acetoacetate, activated ester, imidoester, maleimide, p-nitrophenyl ester, activated halide, anhydride, carbonyl imidazole, epoxide, alkylhalide, and isocyanate. Aldehyde groups form hydrolytically unstable imine bonds when reacted with primary amine groups, which aids in the degradation of crosslinked aminated dextran microspheres prepared using crosslinking agents having aldehyde functional groups. This hydrolytic degradation is enhanced at acidic pH. Crosslinking agents containing at least two of these functional groups include, but are not limited to dialdehydes, such as glutaraldehyde and genipin; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; and di-imidoesters, such as dimethy 3,3-dithiopropionimidate. Particularly useful for medical applications are crosslinking agents that are highly biocompatible, i.e., non-cytoxic, such as genipin and di-imidoesters.

In one embodiment, the crosslinking agent is selected from the group consisting of glutaraldehyde, genipin, and dimethy 3,3-dithiopropionimidate.

In one embodiment, the crosslinking agent is genipin. Genipin (CAS No. 6902-77-8) is a natural crosslinking agent that has a low acute toxicity, and is therefore, ideally suited for use in the preparation of degradable, crosslinked aminated dextran microspheres for medical applications. In aqueous solution, genipin exists in two equilibrium conformations, one of which exposes two aldehyde groups. After reaction with a primary amine to form an imine bond, the product undergoes a cyclization, giving a structure that does not contain imine bonds, so that the crosslinked moiety and thus the resulting microspheres do not degrade hydrolytically.

### Additional Crosslinkable Components

The degradable, crosslinked aminated dextran microspheres may also comprise at least one additional crosslinkable component to alter the properties of the microspheres, for example to make the microspheres more rigid or to increase cell attachment. The additional crosslinkable component contains at least two primary amine groups and is co-crosslinked with the aminated dextran by the crosslinking agent. Suitable examples of additional crosslinkable components include, but are not limited to, other aminated polysaccharides such as aminated derivatives of: carboxymethyldextran, starch, agar, cellulose, hydroxyethylcellulose, carboxymethylcellulose, pullulan, inulin, levan, agarose, and hyaluronic acid; chitosan; and proteins such as gelatin.

In one embodiment, the additional crosslinkable component is gelatin. Gelatin acts as a cell attachment factor, which may improve anchoring of the microspheres to the surrounding tissue, thereby preventing migration of the microspheres from the intended site. Gelatins are water-soluble proteins that are obtained from animal tissues, such as bone and skin, by acid or alkaline treatment. Gelatins suitable for use in the invention include, but are not limited to, gelatins obtained from bovine skin, porcine skin, and fish skin. Gelatins can be obtained commercially from chemical companies such as Sigma-Aldrich, for example.

### Preparation of Degradable, Crosslinked Aminated Dextran Microspheres

The degradable, crosslinked aminated dextran microspheres disclosed herein may be prepared using methods known in the art. For example, the degradable, crosslinked aminated dextran microspheres can be prepared using a suspension crosslinking method. In this method, an aqueous solution comprising aminated dextran is prepared by adding at least one aminated dextran to water to give a concentration of about 3% to about 50% by weight, more particularly about 3% to about 30% by weight, and more particularly about 5% to about 15% by weight, relative to the total weight of the solution. Mixtures of different aminated dextrans, having different average molecular weights and/or different amine substitution levels, may also be used. If a mixture of different aminated dextrans is used, the total concentration of the aminated dextrans is about 3% to about 50% by weight, more particularly about 3% to about 30% by weight, and more particularly about 5% to about 15% by weight, relative to the total weight of the solution. The aqueous solution comprising aminated dextran may also comprise at least one additional crosslinkable component, as described above. If an additional crosslinkable component, as described above, is used, the amount of the additional crosslinkable component may be about 20% to about 50% by weight relative to the weight of aminated dextran, more particularly about 30% to about 40% by weight relative to the weight of aminated dextran.

The aqueous solution comprising aminated dextran may further comprise various additives depending on the intended application. The amount of the additive used depends on the particular application and may be readily determined by one skilled in the art using routine experimentation. For example, the aqueous solution comprising aminated dextran may optionally include at least one pH modifier to adjust the pH of the solution. Suitable pH modifiers are well known in the art. The pH modifier may be an acidic or basic compound. Examples of acidic pH modifiers include, but are not limited to, carboxylic acids, inorganic acids, and sulfonic acids. Examples of basic pH modifiers include, but are not limited to, hydroxides, alkoxides, nitrogen-containing compounds other than primary and secondary amines, and basic carbonates and phosphates.

The aqueous solution comprising aminated dextran may optionally include at least one pharmaceutical drug or therapeutic agent. Suitable drugs and therapeutic agents are well known in the art (for example see the United States Pharmacopeia (USP), Physician's Desk Reference (Thomson Publishing), The Merck Manual of Diagnosis and Therapy 18th ed., Mark H. Beers and Robert Berkow (eds.), Merck Publishing Group, 2006; or, in the case of animals, The Merck Veterinary Manual, 9th ed., Kahn, C. A. (ed.), Merck Publishing Group, 2005). Nonlimiting examples include anti-inflammatory agents, for example, glucocorticoids such as prednisone, dexamethasone, budesonide; non-steroidal anti-inflammatory agents such as indomethacin, salicylic acid acetate, ibuprofen, sulindac, piroxicam, and naproxen; fibrinolytic agents such as a tissue plasminogen activator and streptokinase; anti-coagulants such as heparin, hirudin, ancrod, dicumarol, sincumar, iloprost, L-arginine, dipyramidole and other platelet function inhibitors; antibodies; nucleic acids; peptides; hormones; growth factors; cytokines; chemokines; clotting factors; endogenous clotting inhibitors; antibacterial agents; antiviral agents; antifungal agents; anti-cancer agents; cell adhesion inhibitors; healing promoters; vaccines; thrombogenic agents, such as thrombin, fibrinogen, homocysteine, and estramustine; radio-opaque compounds, such as barium sulfate and gold particles and radiolabels.

An organic solution can be a suitable dispersion medium in the suspension crosslinking method for microsphere preparation. A suitable organic solution comprises a water-immiscible organic solvent, which may be any halogenated aprotic polar solvent, such as 1,2-dichloroethane. The organic solution comprises a viscosity modifying component that provides a surface tension that allows droplet formation in the aqueous/organic suspension formed during the suspension crosslinking method. This viscosity modifying component is called a “protecting colloid”. A variety of natural and synthetic compounds soluble in organic media may be used as a protecting colloid, including, but not limited to, cellulose derivatives, polyacrylates (such as polyacrylic acid and polymethacrylic acid), polyalkylene glycols such as polyethylene glycol, partially hydrolyzed polyvinyl alcohol and other polyols, guar gum, and agar gum. Particularly useful are organic soluble cellulose ethers such as methyl cellulose, hydroxymethyl cellulose, hydroxyethyl cellulose, ethylhydroxyethyl cellulose, hydroxypropyl cellulose, ethyl cellulose, and benzyl cellulose; as well as organic soluble cellulose esters such as cellulose acetate, cellulose butylate, cellulose acetate butylate, cellulose propionate, cellulose butyrate, cellulose acetate propionate, cellulose acetate butyrate, and cellulose acetate phthalate. In one embodiment, the protecting colloid is cellulose acetate butyrate. The amount of the protecting colloid in the organic solution is sufficient to reduce microdroplet coalescence in the aqueous/organic suspension, and is generally between about 0.5% and about 5% by weight of the organic solution.

The aqueous solution comprising aminated dextran and the organic solution are combined and mixed vigorously to form an aqueous/organic mixture. The order of combining the two solutions is not critical to the present invention. Specifically, the aqueous solution may be added to the organic solution, the organic solution may be added to the aqueous solution, or the two solutions may be combined simultaneously. Typically, however, the aqueous solution comprising aminated dextran is added to the organic solution. The two solutions may be combined in various volume ratios. For example, the ratio of the volume of the aqueous solution to the volume of the organic solution may be about 1 to 10, more particularly about 1 to 4. During the combination of the two solutions, the resulting mixture is agitated at a rate capable of forming a uniform suspension from the two solutions. Agitation may be by any method that thoroughly mixes the two solutions, such as shaking or stirring, for example. Typically, the mixture of the two solutions is stirred at a rotation speed of about 450 to 650 rpm for 10 to 20 minutes. After this time, a solution comprising at least one crosslinking agent in water or in a water-miscible organic solvent, such as dimethyl sulfoxide, is added to the aqueous/organic mixture and the resulting mixture is stirred vigorously for a time sufficient to form microspheres, e.g., 4 to 12 hours.

The size of the microspheres may be controlled by adjusting the amount of aminated dextran used and/or the stirring rate. Specifically, increasing the amount of aminated dextran increases the size of the microspheres, whereas increasing the stirring rate decreases the size of the microspheres. The conditions required to obtain microspheres of the desired size range can be readily determined by one skilled in the art using routine experimentation.

In one embodiment, the weight percent of the crosslinking agent relative to the weight of aminated dextran is less than 10%. In another embodiment, the weight percent of the crosslinking agent relative to the weight of aminated dextran is about 1% to about 5%.

In the next step in the suspension crosslinking method, a cold dehydrating solvent, such as acetone, methanol, ethanol, propanol, or butanol, is added to the aqueous/organic mixture and the mixture is stirred in an ice bath to dehydrate the microspheres. The microspheres are collected using means known in the art, such as centrifugation or filtration. The collected microspheres may be washed with cold dehydrating solvent one or more times, and then dried using methods known in the art, such as air-drying, heating, vacuum, and the like.

The crosslinked aminated dextran microspheres disclosed herein may also be produced using any of the methods described by Soppimath et al. (Journal of Controlled Release, 70:1-20, 2001).

The crosslinked aminated dextran microspheres disclosed herein comprise at least one aminated dextran containing primary amine groups and at least one crosslinking agent wherein the aminated dextran and the crosslinking agent are crosslinked through covalent bonds formed between the primary amine groups of the aminated dextran and the functional groups of the crosslinking agent. If the functional groups on the crosslinking agent are aldehyde groups, the covalent bonds may be imine, aminal or hemiaminal bonds. The crosslinked aminated dextran microspheres do not swell significantly when rehydrated in an aqueous medium. The particle size of the microspheres is about 10 microns to about 750 microns, more particularly about 10 microns to about 400 microns, more particularly about 10 microns to about 250 microns, and more particularly about 10 microns to about 125 microns. A heterogeneous size mixture of microspheres may be separated into microsphere samples of specific size ranges, if desired, for specific applications. Microspheres may be separated by methods such as fluidized bed separation and sieving, also called screen filtering. Particularly useful is sieving through a series of sieves appropriate for recovering samples containing microspheres of desired sizes.

The crosslinked aminated dextran microspheres disclosed herein are biocompatible in that they lack cytotoxicity. Additionally, the microspheres may degrade enzymatically in vivo into low molecular weight, soluble polymer, which is small enough to be easily excreted from the body, by the action of enzymes present in the body of mammals. Additionally, microspheres containing hydrolytically unstable bonds (e.g., imine bonds) may also degrade hydrolytically. These properties make the microspheres ideally suited for use as a temporary embolic material.

### Embolization Using the Degradable Crosslinked Aminated Dextran Microspheres

For use in embolization, sterility of the microspheres is an important factor. The degradable, crosslinked aminated dextran microspheres disclosed herein may be sterilized using methods known in the art such as gamma irradiation, ethylene oxide sterilization, or sterilization using ultraviolet light. For embolization, the microspheres are typically used in the form of an embolization suspension which comprises the microspheres in a biocompatible carrier. Suitable biocompatible carriers for use herein include, but are not limited to, dimethylsulfoxide (DMSO), Ethiodol®, MD-76®, Omnipaque™, Visipaque™, and mineral oil. Ethiodol®, MD-76®, Omnipaque™, and Visipaque™, are contrast agents typically used in medical intravascular arteriography or lymphography procedures. Ethiodol® contains iodine organically combined with ethyl esters of the fatty acids of poppyseed oil and is available from SAVAGE Laboratories® (Melville, N.Y.). MD-76® is an aqueous solution of diatrizoate meglumine (CAS No. 131-49-7, 66 wt %) and diatrizoate sodium (CAS No. 737-31-5, 10 wt %) buffered with monobasic sodium, with a pH of 6.5 to 7.7, having organically bound iodide to provide for radiological visualization. MD-76® is manufactured by Mallinckrodt Inc. (St. Louis, Mo.). Omnipaque™ and Visipaque™ (GE Healthcare, Inc., Princeton, N.J.) are aqueous solutions containing Iodixanol (CAS No. 92339-11-2).

The microsphere concentration in the embolization suspension varies depending on the carrier used and the size catheter to be used for administering the suspension, which in turn depends on the size of the vasculature to be embolized. In addition, the size of the microspheres affects the concentration used, where samples of different sized microspheres may be prepared, for example by sieving, as described above. The specific size and concentration of microspheres, as well as the desired carrier, may be chosen by one skilled in the art for the particular embolization treatment to be performed.

The embolization suspension containing the degradable, crosslinked aminated dextran microspheres disclosed herein is administered to a mammal for embolization, as is known to one skilled in the art, for example as described in “Uterine Artery Embolization and Gynecologic Embolotherapy”, Spies and Pelage, 2005 ISBN: 0-7817-4532-2 and in “Vascular and Interventional Radiology: Principles and Practice”, Bakal et al., 2002 ISBN: 0-86577-678-4. Administration of the embolization suspension containing the microspheres is generally by passage through a catheter or needle into the vasculature of the mammal such that the microspheres reach a target site, where they agglomerate to form an occlusion. The occlusion effectively blocks the blood flow distal to the occlusion site. The occlusion site may be any target site where, for medical treatment, it is desired to block the flow of blood. For example, the occlusion site may be in a blood vessel that feeds a tumor such as a uterine fibroid or a cancerous tumor, in an arteriovenous malformation, or in a blood vessel where the blood is not contained, such as in the case of a stomach ulcer or injury. Preoperative embolization may also be performed to stop blood flow to a region targeted for surgery.

In addition to forming an occlusion, the degradable, crosslinked aminated dextran microspheres used in embolization may also be prepared such that they are able to deliver medications, such as pharmaceutical drugs or therapeutic agents. The medication may be loaded into or coated onto the microspheres using various methods known in the art. For example, the microspheres may be imbibed with the medication by hydrating dried microspheres in a medium containing the medication and allowing it to soak into the microspheres. The microspheres may then be dried by removing water by washing with a dehydrating solvent, as described above. Additionally, the medication may be coated onto the microspheres using methods such as spraying, immersion, and the like. The medication may also be directly incorporated into the microspheres during their preparation by adding the medication to the aqueous solution comprising aminated dextran, as described above. Following delivery of the microspheres containing the medication to the target site, the medication is released over time as the microspheres are in contact with body fluids. For example, anti-cancer drugs may be delivered by microspheres forming an occlusion in proximity to a cancerous tumor. Delivery in embolizing microspheres of agents such as anti-angiogenic factors, anti-inflammatory drugs, analgesics, and local anesthetics provide additional treatment to the physical blockage of embolization. Additional pharmaceutical drugs and therapeutic agents that may be delivered in the degradable, crosslinked aminated dextran microspheres are described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

Chemicals used in the Examples were obtained from Sigma-Aldrich (St. Louis, Mo.) and were used without further purification unless otherwise indicated.

### Reagent Preparation

### Preparation of Aminated Dextran:

An aminated dextran containing primary amine groups was prepared using a two step procedure. In the first step, dextran was reacted with epichlorohydrin in the presence of an acid catalyst to give 3-chloro-2 hydroxypropyl dextran. In the second step, the 3-chloror-2-2hydroxypropyl dextran was reacted with aqueous ammonia to give 3-amino-2-hydroxypropyl dextran.

(i) Preparation of 3-chloro-2-hydroxypropyl Dextran by the Following Reaction

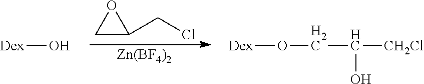

In the first step, 20 g of dextran having an average molecular weight of 9-11 kDa was dissolved in a solution containing 30 mL of a 25% aqueous solution of Zn(BF₄)₂ and 20 mL of water. Epichlorohydrin (100 mL) was added to the dextran solution with vigorous stirring and the mixture was allowed to react for 3 hours at 80° C., and subsequently overnight at room temperature. The resulting product was precipitated by pouring the solution dropwise into acetone. The precipitate was filtered, and dried under vacuum. The 3-chloror-2-2-hydroxypropyl dextran was obtained in 75% yield.

(ii) Preparation of 3-amino-2-hydroxypropyl dextran by the following reaction

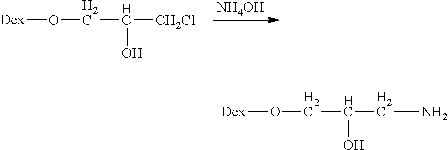

The 3-chloro-2-2-hydroxypropyl dextran (4.1 g) was dissolved in a solution containing 60 mL of water and 20 mL of aqueous ammonia (28 wt %). This solution was stirred for 2 days at room temperature and then poured dropwise into 1.0 L of methanol. The resulting precipitate of 3-amino-2-hydroxypropyl dextran was filtered, washed with acetone, and dried under reduced pressure. The chlorine atom was successfully replaced by an amino group as confirmed by the ninhydrin test (dark blue color) and proton NMR. ¹H-NMR (D₂O) δ 2.80-3.0 ppm. (m, 2-hydroxypropyl ether c) δ 3.30-4.20 ppm. (m, 2-hydroxypropyl ether a, dextran C₂-C₆) δ 5.0-5.4 ppm. (m, b, dextran C1).

The yield of 3-amino-2-hydroxypropyl dextran was 75%. The 3-amino-2-hydroxypropyl dextran product was analyzed using size exclusion chromatography (SEC), which indicated that no significant degradation or crosslinking of the dextran had occurred during the reactions.

### Examples 1-3

### Preparation of Degradable Microspheres of Aminated Dextran Crosslinked with Genipin

The purpose of these Examples was to prepare degradable microspheres by crosslinking aminated dextran with various amounts of genipin crosslinking agent.

An aqueous solution of 3-amino-2-hydroxypropyl dextran (10 mL of a 10% stock solution) was mixed with different volumes of genipin solution (50 mg/mL in DMSO) at genipin to aminated dextran ratios of 1, 2, 3 and 5 wt % (see Table 1). In a typical preparation, the reaction mixture was incubated for 20 hours, after which the mixture (dark blue) was added to 40 mL of poly(propylene glycol) in a 100 mL flask. The resulting mixture was stirred at 450 rpm for 2 hours using a paddle mixer at 50° C., then cooled to 4° C. for 2 hours without interrupting the stirring. The resulting blue crosslinked microspheres were washed with excess acetone and dried under vacuum.

The morphology and size of the genipin-crosslinked aminated dextran microspheres was characterized using scanning electron microscopy (SEM). The mean particle size of the microspheres, determined from a sample of 200 microspheres, is given in Table 1. When the wt % of genipin relative to the aminated dextran was 10%, microspheres were not formed; instead an amorphorsous hydrogel was obtained.

TABLE 1
Genipin-Crosslinked Aminated Dextran Microspheres

| | Wt % of Genipin | |
|---|---|---|
| | Relative to Aminated | Mean Particle Size |
| Example | Dextran | (μm) |
| 1 | 1% | 8-15 |
| 2 | 3% | 15-30 |
| 3 | 5% | 15-30 |

### Example 4

### Cytotoxicity Testing of Genipin-Crosslinked Aminated Dextran Microspheres

The purpose of this Example was to demonstrate the safety of genipin-crosslinked aminated dextran microspheres in an in vitro test.

The testing was done using NIH3T3 mouse fibroblast cell cultures according to ISO10993-5:1999. The NIH3T3 mouse fibroblast cells were obtained from the American Type Culture Collection (ATCC; Manassas, Va.) and were grown in Dulbecco's modified essential medium (DMEM), supplemented with 10% fetal calf serum.

Samples containing 1, 5 and 10 mg of the microspheres described in Examples 1-3 were placed on the bottom of the wells of a polystyrene tissue culture plate. The plate was then sterilized under UV light overnight. The next day, cells (50,000-100,000 NIH3T3 cells) were deposited in the culture plates and the plates were incubated for 24-48 hours.

The growth of the NIH3T3 cells in direct contact with the microspheres was monitored. The cells formed a confluent layer on the bottom of the well and appeared to cover parts of the surface of the microspheres, which suggests that the microspheres are non-cytotoxic.

### Example 5

### Enzymatic Degradation of Genipin-Crosslinked Aminated Dextran Microspheres

The purpose of this Example was to demonstrate that the genipin-crosslinked aminated dextran microspheres are enzymatically degraded, resulting in low molecular weight, soluble polymer.

Genipin-crosslinked aminated dextran microspheres (3 wt % genipin, as described in Example 2) were added at 2 wt % to 0.1 M potassium citrate buffer, pH 6.0. Aliquots (0.9 mL) of these suspensions were mixed with 0.1 mL of endodextranase (MP Biochemicals, Solon, Ohio, 500 units/mg) enzyme solution (50 units/mL) in test tubes. For the negative control, 0.1 mL of deionized water was added in place of the enzyme solution. The tubes were incubated at 37° C. and samples of the microsphere-dextranase mixtures were taken and quenched at timed intervals up to 19 hours by placing the samples in a water bath at 100° C. for 15 min. Then, 100 μL of each inactivated sample was analyzed using size exclusion chromatography (SEC) using low molecular weight dextran standards (American Polymer Standards, Mentor, Ohio) as calibration standards. After 19 hours, the degradation product from the microspheres had an average molecular weight of about 2,000 Da, which is small enough to be easily excreted from the body.

### Examples 6-8

### Preparation of Degradable Microspheres of Crosslinked Aminated Dextran and Gelatin

The purpose of these Examples was to prepare degradable microspheres by crosslinking aminated dextran and gelatin together with various crosslinking agents. Gelatin served as an additional crosslinable component. The microspheres were prepared with a suspension crosslinking method using a discontinuous aqueous phase dispersed in a continuous 1,2-dichloroethane organic phase.

Equal volumes (3 mL) of an aqueous aminated dextran solution (12 wt %) and an aqueous gelatin (from porcine skin) solution (12 wt %) were added to 44 mL of a solution of 4 wt % cellulose acetate butyrate (Mₙ of approximately 65,000 Da) in 1,2-dichloroethane. This mixture was stirred for 20 min at 450 rpm using an overhead stirrer. Solutions of the crosslinking agents, as shown in Table 2, were used as follows. For the glutaraldehyde crosslinking agent, 2 mL of a solution containing glutaraldehyde (50% in water) was added to 2 mL of 1,2-dichloroethane and vortexed. After vortexing, the mixture was allowed to separate into an aqueous phase and an organic phase. The organic phase was taken and added to the aqueous solution containing the aminated dextran and the gelatin, and the resulting mixture was stirred for 12 hours at 450 rpm. For the genipin crosslinking agent, 4.0 mL of a solution containing 50 mg/mL of genipin in dimethyl sulfoxide (DMSO) was added to the aqueous solution containing the dextran amine and the gelatin, and the resulting mixture was stirred for 12 hours at 450 rpm. For the dimethyl 3,3′-dithiopropionimidate dihydrochloride crosslinking agent, 2.0 mL of an aqueous solution containing 75 mg of the crosslinking agent was added to the aqueous solution containing the aminated dextran and the gelatin, and the resulting mixture was stirred for 12 hours at 450 rpm. Then, 50 mL of cold acetone was added to the flasks containing the mixtures, the flasks were placed in an ice bath, and stirring was continued for another hour to dehydrate the microspheres. The microspheres were collected from each flask by centrifugation, resuspended in 50 mL of acetone, and stirred in an ice bath at 50 to 100 rpm. This washing procedure was repeated to give a total of four washings and then the microspheres were left to dry at room temperature overnight.

The morphology and size of the crosslinked aminated dextran/gelatin microspheres was characterized using scanning electron microscopy (SEM). The mean particle size of the microsphere is given in Table 2. SEM of thin cross sections of the microspheres indicated that the interior of the microspheres had a sponge-like core.

TABLE 2
Crosslinked Aminated Dextran/Gelatin Microspheres

| | | Mean Particle Size |
|---|---|---|
| Example | Crosslinking Agent | (μm) |
| 6 | glutaraldehyde | 125 |
| 7 | genipin | 100 |
| 8 | dimethyl 3,3′-dithiopropionimidate | 75 |
| | dihydrochloride | |

### Example 9

### Cytotoxicity Testing of Crosslinked Aminated Dextran/Gelatin Microspheres

The purpose of this Example was to demonstrate the safety of the crosslinked aminated dextran/gelatin microspheres in an in vitro test.

The cytotoxicity of the crosslinked aminated dextran/gelatin microspheres from Examples 6-8 was tested using NIH3T3 mouse fibroblast cell cultures using the procedure described in Example 4. All the microspheres were found to be noncytotoxic by this method.

What is claimed is:

1. A composition comprising the reaction products obtained by the reaction of:
a) at least one aminated dextran containing primary amine groups, said at least one aminated dextran having a weight-average molecular weight of about 1,000 to about 1,000,000 Daltons and an amine substitution level of about 1% to about 65%; and
b) at least one crosslinking agent containing at least two functional groups capable of reacting with the primary amine groups of the aminated dextran;
wherein:
(i) said at least one aminated dextran and said at least one crosslinking agent are crosslinked through covalent bonds formed between the primary amine groups of the aminated dextran and the functional groups of the crosslinking agent; and
(ii) the composition is degradable and in the form of microspheres having a size of about 10 microns to about 750 microns in diameter.

2. The composition according to claim 1 wherein the functional groups contained on the crosslinking agent are selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, and isocyanate.

3. The composition according to claim 1 wherein the crosslinking agent is selected from the group consisting of glutaraldehyde, genipin, and dimethy 3,3-dithiopropionimidate.

4. The composition according to claim 3 wherein the crosslinking agent is genipin.

5. The composition according to claim 1 wherein the microspheres have a size of about 10 microns to about 125 microns in diameter.

6. The composition according to claim 1 wherein said composition further comprises an additional crosslinkable component that contains at least two primary amine groups.

7. The composition according to claim 6 wherein said additional crosslinkable component is gelatin.

8. The composition according to claim 1 wherein the composition comprises the crosslinking agent at less than 10 weight percent relative to the weight of the aminated dextran.

9. The composition according to claim 8 wherein the composition comprises the crosslinking agent at about 1 weight percent to about 5 weight percent relative to the weight of the aminated dextran.

10. A method for embolization in a mammal comprising administering into the vasculature of said mammal a composition comprising:
a) at least one aminated dextran containing primary amine groups, said at least one aminated dextran having a weight-average molecular weight of about 1,000 to about 1,000,000 Daltons and an amine substitution level of about 1% to about 65%; and
b) at least one crosslinking agent containing at least two functional groups capable of reacting with the primary amine groups of the aminated dextran;
wherein:
(i) said at least one aminated dextran and said at least one crosslinking agent are crosslinked through covalent bonds formed between the primary amine groups of the aminated dextran and the functional groups of the crosslinking agent; and
(ii) the composition is in the form of degradable, microspheres having a size of about 10 microns to about 750 microns in diameter.

11. The method according to claim 10 wherein the functional groups contained on the crosslinking agent are selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, and isocyanate.

12. The method according to claim 10 wherein the crosslinking agent is selected from the group consisting of glutaraldehyde, genipin, and dimethy 3,3-dithiopropionimidate.

13. The method according to claim 12 wherein the crosslinking agent is genipin.

14. The method according to claim 10 wherein the microspheres have a size of about 10 microns to about 125 microns in diameter.

15. The method according to claim 10 wherein said composition further comprises an additional crosslinkable component that contains at least two primary amine groups.

16. The method according to claim 15 wherein said additional crosslinkable component is gelatin.

17. The method according to claim 10 wherein the composition comprises the crosslinking agent at less than 10 weight percent relative to the weight of the aminated dextran.

18. The method according to claim 17 wherein the composition comprises the crosslinking agent at about 1 weight percent to about 5 weight percent relative to the weight of the aminated dextran.
